(19) United States
(12) Patent Application Publication
(10) Pub. No.: US 2024/0139155 A1
(43) Pub. Date: May 2, 2024

(54) COMPOUNDS FOR TREATING TNBC

(71) Applicant: Purdue Pharma L.P., Stamford, CT (US)

(72) Inventor: Thomas Jorg Mehrling, Basel (CH)

(73) Assignee: Purdue Pharma L.P., Stamford, CT (US)

(21) Appl. No.: 18/244,913

(22) Filed: Sep. 12, 2023

Related U.S. Application Data
(63) Continuation of application No. 17/885,696, filed on Aug. 11, 2022, now Pat. No. 11,786,509.
Application No. 17/885,696 is a continuation of application No. 16/621,896, filed on Dec. 12, 2019, now Pat. No. 11,413,276.

(30) Foreign Application Priority Data
Jun. 13, 2017 (GB) 1709406.1

(51) Int. Cl. A61K 31/4184; A61P 35/00

(52) U.S. Cl. CPC A61K 31/4184; A61P 35/00; A61K 9/0019

(57) ABSTRACT

There is provided tinostamustine or a pharmaceutically acceptable salt thereof for use in the treatment of triple negative breast cancer (TNBC) to a patient in need thereof.

### TECHNICAL FIELD

The present invention relates to methods of treating cancer, particularly triple negative breast cancer (TNBC).

### BACKGROUND OF THE INVENTION

Cancer is one of the most life threatening diseases. Cancer is a condition in which cells in a part of the body experience out-of-control growth. According to latest data from American Cancer Society, it is estimated there will be 1.69 million new cases of cancer in USA in 2017. Cancer is the second leading cause of death in the United States (second only to heart disease) and will claim more than 601,000 lives in 2017. In fact, it is estimated the average lifetime risk of developing cancer is 40.8% for American males and 37.5% for American women. Therefore cancer constitutes a major public health burden and represents a significant cost in the United States. These figures are reflected elsewhere across most countries globally, although the types of cancer and relative proportions of the population developing the cancers vary depending upon many different factors such including genetics and diet.

For decades surgery, chemotherapy, and radiation were the established treatments for various cancers. Patients usually receive a combination of these treatments depending upon the type and extent of their disease. But chemotherapy is the most important option for cancer patients when surgical treatment (i.e. the removal of diseased tissue) is impossible. While surgery is sometimes effective in removing tumours located at certain sites, for example, in the breast, colon, and skin, it cannot be used in the treatment of tumours located in other areas, such as the backbone, nor in the treatment of disseminated hematological cancers including cancers of the blood and blood-forming tissues (such as the bone marrow). Such cancers include multiple myeloma, lymphoma and leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. It is used most often in the treatment of breast, lung, and testicular cancer. One of the main causes of failure in chemotherapy is the development of drug resistance by the cancer cells, a serious problem that may lead to recurrence of disease or even death. Thus, more effective cancer treatments are needed.

Solid tumours are an abnormal mass of tissue that usually does not contain cysts or liquid areas. Solid tumours may be benign (not cancer), or malignant (cancer). Different types of solid tumours are named for the type of cells that form them. Examples of solid tumours are carcinomas and sarcomas. The four most common cancers occurring worldwide are all solid tumours, namely lung, breast, bowel and prostate cancer. These four solid tumour cancers account for around 4 in 10 of all cancers diagnosed worldwide.

According to the National Cancer Institute, there were an estimated 252,170 new cases of and 40,610 deaths due to breast cancer (women only) in the US in 2017.

Breast cancer is the most common noncutaneous cancer in US women, with an estimated 61,000 cases of in situ disease and 246,660 cases of invasive disease in 2016. Thus, fewer than one of six women diagnosed with breast cancer die of the disease. By comparison, it is estimated that about 71,280 American women will die of lung cancer in 2017. Men account for 1% of breast cancer cases and breast cancer death.

Breast cancer is not one form of cancer but consists of many different subtypes of cancers. These subtypes of breast cancer are generally diagnosed based upon the presence (or lack of) three receptors: estrogen receptors, progesterone receptors and human epidermal growth factor receptor 2 (HER2). Around 10-20% of breast cancers test negative for both estrogen and prosegrerone receptors and have HER2 gene amplification, and are known as triple negative breast cancer (TNBC). TNBC tends to be more aggressive than other types of breast cancer and it is more likely to metastasise or recur.

Given the prevalence of solid tumours including breast cancer, there is therefore a need for new effective chemotherapeutic treatments.

In WO-A-2010/085377, the compound of formula I below is disclosed. It is a first-in-class dual-functional alkylating-HDACi fusion molecule which potently inhibits HDAC-regulated pathways.

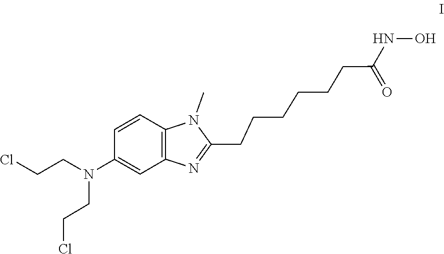 [SMILES: Cn1c(CCCCCCC(=O)NO)nc2cc(N(CCCl)CCCl)ccc21]

Biological assays showed that the compound of formula I potently inhibits HDAC enzyme (HDAC1 IC₅₀ of 9 nM). The compound of formula I has an INN of tinostamustine and is also known in the art as EDO-S101. It is an AK-DAC (a first-in-class alkylating deacetylase molecule) that, in preclinical studies, has been shown to simultaneously improve access to the DNA strands within cancer cells, break them and block damage repair.

### SUMMARY OF THE INVENTION

In a first aspect of the present invention there is provided tinostamustine or a pharmaceutically acceptable salt thereof for use in the treatment of triple negative breast cancer (TNBC) in a patient in need thereof, wherein:
the TNBC is selected from: basal-like 1 (BL1) subtype; and/or
the TNBC is BRCA-1 wildtype.

It has surprisingly been discovered that tinostamustine or a pharmaceutically acceptable salt thereof is particularly effective in the treatment of TNBC, with activity data showing strong sensitivity to this compound. Thus, the need for a new and effective treatment of TNBC is met by the present invention.

In a further aspect of the present invention there is provided tinostamustine or a pharmacologically acceptable salt thereof for use as a monotherapy in the treatment of triple negative breast cancer (TNBC) in a patient in need thereof. Optionally:
the TNBC is selected from: basal-like 1 (BL1) subtype; and/or
the TNBC is BRCA-1 wildtype.

In a further aspect of the present invention there is provided the use of tinostamustine or a pharmaceutically acceptable salt thereof for the manufacture of a medicament for the treatment of triple negative breast cancer (TNBC) in a patient in need thereof, wherein:
the TNBC is selected from: basal-like 1 (BL1) subtype; and/or
the TNBC is BRCA-1 wildtype.

In a further aspect of the present invention there is provided the use of tinostamustine or a pharmaceutically acceptable salt thereof as a monotherapy for the manufacture of a medicament for the treatment of triple negative breast cancer (TNBC) in a patient in need thereof. Optionally:
the TNBC is selected from: basal-like 1 (BL1) subtype; and/or
the TNBC is BRCA-1 wildtype.

In a further aspect of the present invention there is provided a method of treating triple negative breast cancer (TNBC) in a patient in need thereof comprising administering to said patient an effective amount of tinostamustine or a pharmaceutically acceptable salt thereof, wherein:
the TNBC is selected from: basal-like 1 (BL1) subtype; and/or
the TNBC is BRCA-1 wildtype.

In a further aspect of the present invention there is provided a method of treating triple negative breast cancer (TNBC) in a patient in need thereof comprising administering to said patient an effective amount of tinostamustine or a pharmaceutically acceptable salt thereof as a monotherapy. Optionally:
the TNBC is selected from: basal-like 1 (BL1) subtype; and/or
the TNBC is BRCA-1 wildtype.

In a further aspect of the present invention there is provided a kit comprising tinostamustine or a pharmaceutically acceptable salt thereof together with instructions for treating triple negative breast cancer (TNBC) in a patient in need thereof, wherein:
the TNBC is selected from: basal-like 1 (BL1) subtype; and/or
the TNBC is BRCA-1 wildtype.

In a further aspect of the present invention there is provided a kit comprising tinostamustine or a pharmaceutically acceptable salt thereof together with instructions for treating triple negative breast cancer (TNBC) as a monotherapy in a patient in need thereof. Optionally:
the TNBC is selected from: basal-like 1 (BL1) subtype; and/or
the TNBC is BRCA-1 wildtype.

In a further aspect, tinostamustine or a pharmaceutically acceptable salt thereof may be administered in combination with one or more further agent(s). The further agent(s) may be DNA repair inhibitors. The further agent(s) may be DNA damage response (DDR) inhibitors. The further agent may be PARP inhibitors. The PARP inhibitor may be olaparib.

The further agent may be carboplatin.

When used in combination, tinostamustine or a pharmaceutically acceptable salt thereof and further agent(s) can be administered concurrently, sequentially or separately. In an embodiment, the agents are administered concurrently. In a further embodiment, the agents are administered sequentially. In a yet further embodiment, the agents are administered separately.

In a further aspect, there is provided a combination comprising tinostamustine or a pharmaceutically acceptable salt thereof and a further agent(s) selected from DNA repair inhibitors, DDR inhibitors, PARP inhibitors, or olaparib.

Combinations of tinostamustine or a pharmaceutically acceptable salt thereof and a further agent(s) selected from DNA repair inhibitors, DDR inhibitors, PARP inhibitors, or olaparib have been found to be particularly effective in the treatment of cancers, such as TNBC, such that they are highly promising in efforts to address the problems of finding more effective treatments of cancer.

Tinostamustine or a pharmaceutically acceptable salt thereof, and further agent(s) can be administered concurrently, sequentially or separately. In an embodiment, the agents are administered concurrently. In a further embodiment, the agents are administered sequentially. In a yet further embodiment, the agents are administered separately.

In a further aspect, there is provided a pharmaceutical composition comprising a pharmaceutically acceptable carrier and a combination according to the present invention.

In a further aspect, there is provided a kit comprising a combination according to the present invention and optionally instructions for treating a patient.

In a further aspect, there is provided a combination according to the present invention for use as a medicament.

In a further aspect, there is provided a combination according to the present invention for use in the treatment of cancer.

In a further aspect, there is provided the use of a combination according to the present invention for the manufacture of a medicament for the treatment of cancer.

In a further aspect, there is provided a method of treating cancer in a patient in need thereof comprising administering a combination according to the present invention.

The following features apply to all aspects of the invention.

The cancer may be TNBC.

The TNBC subtype may be BL1.

The TNBC subtype may be BRCA-1 wildtype.

The TNBC may be relapsed and/or refractory.

The TNBC may be platinum resistant.

The TNBC may be BRCA-1 wildtype.

The cancer may be relapsed and/or refractory.

The cancer may be localized.

The cancer may be metastatic.

The cancer may be advanced.

The cancer may have progressed after at least one line of standard therapy.

One or more further agent(s) may be administered in combination with tinostamustine or a pharmaceutically acceptable salt thereof.

The combination may be tinostamustine or a pharmaceutically acceptable salt thereof and a further agent or agents.

The further agent may be carboplatin.

The further agent(s) may be DNA repair inhibitors.

The further agent(s) may be DDR inhibitors.

The further agent(s) may be PARP inhibitors.

The PARP inhibitor may be olaparib.

The further agents may be carboplatin and a PARP inhibitor.

The DNA repair inhibitors, DDR inhibitors, PARP inhibitors, olaparib may be combined with carboplatin.

The further agents may be carboplatin and olaparib.

### DESCRIPTION OF THE DRAWINGS

FIG. 1a shows a HCC1143 p13 spheroid after 48 h before starting treatment.

FIG. 1b shows the residual cell viability (% solvent control) of the HCC1143 p13 spheroid model for EDO-S101 alone and in combination with carboplatin and/or olaparib after 48 h and 72 h.

### DETAILED DESCRIPTION OF THE INVENTION

In the present application, a number of general terms and phrases are used, which should be interpreted as follows.

The compound of formula I has an INN of tinostamustine and is also known in the art as EDO-S101. The IUPAC name is 7-(5-(bis(2-chloroethyl)amino)-1-methyl-1H-benzo[d]imidazol-2-yl)-N-hydroxyheptanamide.

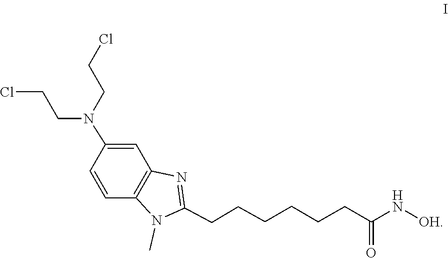 [SMILES: Cn1c(CCCCCCC(=O)NO)nc2cc(N(CCCl)CCCl)ccc21]

“Patient” includes humans, non-human mammals (e.g., dogs, cats, rabbits, cattle, horses, sheep, goats, swine, deer, and the like) and non-mammals (e.g., birds, and the like).

“Pharmaceutically acceptable salts” means salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

In the present invention, the pharmaceutically acceptable salt of tinostamustine may preferably be the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate salt.

It has surprisingly been found that tinostamustine or a pharmaceutically acceptable salt thereof shows surprising efficacy in solid tumours. In particular, it has been found that tinostamustine or a pharmaceutically acceptable salt thereof is useful in the treatment of TNBC.

Breast cancer is not one form of cancer but many different subtypes of cancers. These subtypes of breast cancer are generally diagnosed based upon the presence (or lack of) three receptors: estrogen receptors, progesterone receptors and human epidermal growth factor receptor 2 (HER2). Around 10-20% of breast cancers test negative for both estrogen and progesterone receptors and have HER2 gene amplification, and are known as triple negative breast cancer (TNBC).

The absence of these receptors means that the cancers are unlikely to respond to hormonal therapies, including tamoxifen, anastrozole, exemestane, letrozole and fulvestrant). Triple-negative breast cancers are also unlikely to respond to medications that target HER2, such as trastuzumab or lapatinib.

Triple negative breast cancers also tend to be more aggressive than other types of breast cancer and are more likely to metastasise or recur. Five year survival rates also tend to be lower for triple negative breast cancers. For example, a 2007 study of more than 50,000 women with all stages of breast cancer found that 77% of women with triple-negative breast cancer survived at least 5 years, versus 93% of women with other types of breast cancer. Another study of more than 1,600 women published in 2007 found that women with triple-negative breast cancer had a higher risk of death within 5 years of diagnosis, but not after that time period.

Triple negative breast cancer tends to be higher grade than other types of breast cancer, often scoring grade 3 in their appearance and growth patterns.

Triple negative breast cancer represents a heterogeneous diseases and gene expression analysis has identified distinct subtypes, each displaying a unique biology: basal-like (including basal-like 1 (BL1) and basal-like 2 (BL2) subgroups), mesenchymal (M), and luminal androgen receptor (LAR) subtypes (Lehmann et al Refinement of Triple-Negative Breast Cancer Molecular Subtypes: Implications for Neoadjuvant Chemotherapy Selection; PLOS 1; 2016 https://doi.org/10.1371/journal.pone.0157368).

The BL1 subtype expresses high levels of genes involved in cell proliferation and DNA damage response. Cell lines linked to the BL1 subtype include HCC1143 (BRCA-1 wild type) and HCC1937 (BRCA-1 negative).

Given the prevalence of TNBC and resistance to current therapies, there is a need for new effective chemotherapeutic treatments.

It has been surprisingly shown that tinostamustine or a pharmaceutically acceptable salt is effective in the treatment of TNBC basal-like 1 (BL1) subgroup. It has further been shown that tinostamustine or a pharmaceutically acceptable salt is effective in the treatment of BRCA-1 wildtype breast cancer.

Tinostamustine or a pharmaceutically acceptable salt has also been shown for the first time to be effective as a monotherapy in the treatment of TNBC.

The therapeutically effective amount of tinostamustine or a pharmaceutically acceptable salt administered to the patient is an amount which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect). An effective amount of tinostamustine or a pharmaceutically acceptable salt thereof according to the present invention is believed to be one wherein tinostamustine or a pharmaceutically acceptable salt thereof is included at a dosage range of from 0.3 mg/m² to 300 mg/m² body surface area of the patient or from 60 mg/m² to 150 mg/m² body surface area of the patient. In a preferred embodiment, the dosage range is from 80 mg/m² to 100 mg/m² body surface area of the patient.

The specific therapeutically effective dose level for any particular patient will depend upon a variety of factors including the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

“Metastatic Cancer”. Cancer has the ability to spread within the body. Cancer cells can spread locally by moving into nearby normal tissue. Cancer can also spread regionally, to nearby lymph nodes, tissues, or organs. Cancer can therefore spread to distant parts of the body. When this happens, it is called metastatic cancer (also known as stage IV cancer), and the process by which cancer cells spread to other parts of the body is called metastasis. Thus, in metastasis, cancer cells break away from where they first formed (primary cancer), travel through the blood or lymph system, and form new tumours (metastatic tumours) in other parts of the body.

Metastatic cancer cells have features like that of the primary cancer and not like the cells in the place where the cancer is found. This enables doctors to tell whether a cancer is metastatic. Metastatic cancers are given the same name as the primary cancer. For example, breast cancer that has spread to the lung is called metastatic breast cancer, not lung cancer. It is treated as stage IV breast cancer, not as lung cancer.

Metastatic TNBC refers to a triple negative breast cancer that has metastasised to a new location in the body. The cancer is treated as a stage IV TNBC.

“Advanced Cancer” is a cancer that is not curable but responds to treatment. Disease directed therapy is still very important because it prolongs life. For terminal cancer, therapy cannot prolong survival significantly due to the progressive nature of the disease and palliative care is the main treatment option.

“Monotherapy” according to the present invention means tinostamustine or a pharmaceutically acceptable salt thereof is administered alone as a single agent. i.e. it is not administered in combination with a further drug or drugs. Examples of such drugs include (but are not limited to) proteasome inhibitors, glucocorticoids and/or tyrosine kinase inhibitors. However, a tinostamustine monotherapy may include radiotherapy.

“DNA repair inhibitors” and “DNA damage response (DDR) inhibitors” target cell repair/response pathways and are a promising therapeutic avenue in cancer therapy. An example is the class of poly(ADP-ribose) polymerase (PARP) inhibitors, including olaparib.

In embodiments of the present invention tinostamustine is administered in combination with a further agent or agents. These agents may include DNA repair inhibitors, DDR inhibitors, PARP inhibitors, olaparib and/or carboplatin. In embodiments, the combinations may exclude proteasome inhibitors, glucocorticoids and/or tyrosine kinase inhibitors.

Suitable examples of the administration form of tinostamustine or a pharmaceutically acceptable salt thereof include without limitation oral, topical, parenteral, sublingual, rectal, vaginal, ocular, and intranasal. Parenteral administration includes subcutaneous injections, intravenous, intramuscular, intrasternal injection or infusion techniques. Preferably, tinostamustine or a pharmaceutically acceptable salt thereof is administered parenterally, and most preferably intravenously.

“BRCA-1 wildtype”. BRCA-1 is a gene found on chromosome 17. BRCA-1 is a human gene that produce tumour suppressor proteins. These proteins help repair damaged DNA and, therefore, play a role in ensuring the stability of each cell's genetic material. When either of these genes is mutated, or altered, such that its protein product is not made or does not function correctly, DNA damage may not be repaired properly. As a result, cells are more likely to develop additional genetic alterations that can lead to cancer. Specific inherited mutations in BRCA-1 most notably increase the risk of female breast and ovarian cancers, but they have also been associated with increased risks of several additional types of cancer. People who have inherited mutations in BRCA-1 tend to develop breast and ovarian cancers at younger ages than people who do not have these mutations.

A harmful BRCA-1 mutation can be inherited from a person's mother or father. Each child of a parent who carries a mutation in the gene has a 50% chance (or 1 chance in 2) of inheriting the mutation. The effects of mutations in BRCA-1 are seen even when a person's second copy of the gene is normal.

In contrast, a person who has a BRCA-1 gene which is not mutated has a ‘wildtype’ i.e. non-mutated gene. BRCA-1 wildtype means a patient who has non-mutated BRCA-1 gene.

A woman's lifetime risk of developing breast cancer is greatly increased if she inherits a harmful mutation in BRCA-1. About 12% of women in the general population will develop breast cancer sometime during their lives. By contrast, a recent large study estimated that about 72% of women who inherit a harmful BRCA-1 mutation will develop breast cancer by the age of 80. Like women from the general population, those with harmful BRCA-1 mutations also have a high risk of developing a new primary cancer in the opposite (contralateral) breast in the years following a breast cancer diagnosis. It has been estimated that, by 20 years after a first breast cancer diagnosis, about 40% of women who inherit a harmful BRCA-1 mutation will develop cancer in their other breast. As such, patients having a mutated BRCA-1 gene carry an increased risk of getting cancer.

At the same time, the presence of mutations on the BRCA-1 gene limits DNA repair pathways of BRCA-1 mutant tumours, making these tumours more susceptible to certain therapies including DNA damaging agents like platinum agents. Thus, patients who are BRCA-1 wildtype may have worse prognosis and outcomes.

Preferably, tinostamustine or a pharmaceutically acceptable salt thereof is administered intravenously to the patient in need thereof at a dosage level to the patient in need thereof of from 0.3 mg/m² to 300 mg/m² body surface area of the patient.

Preferably, tinostamustine or a pharmaceutically acceptable salt thereof is administered intravenously to the patient in need thereof at a dosage level to the patient in need thereof of from 60 mg/m² to 150 mg/m² body surface area of the patient.

Preferably, tinostamustine or a pharmaceutically acceptable salt thereof is administered intravenously to the patient in need thereof at a dosage level to the patient in need thereof of from 80 mg/m² to 100 mg/m² body surface area of the patient.

It has been found that in embodiments of the present invention, tinostamustine or a pharmaceutically acceptable salt thereof or medicament comprising the same may preferably be administered to a patient in need thereof on days 1, 8 and 15 of a 28 day treatment cycle or on days 1 and 15 of a 28 day treatment cycle.

Preferably, tinostamustine or a pharmaceutically acceptable salt thereof is administered on days 1 and 15 of a 28 day treatment cycle.

It has been found that in embodiments of the present invention, tinostamustine or a pharmaceutically acceptable salt thereof or medicament comprising the same may preferably be administered to a patient in need thereof over an infusion time of 60 minutes; or an infusion time of 45 minutes; or an infusion time of 30 minutes.

Preferably, tinostamustine or a pharmaceutically acceptable salt thereof is administered over an infusion time of 60 minutes.

In a preferred embodiment, tinostamustine or a pharmaceutically acceptable salt is administered to the patient in need thereof at a dosage level of from 80 mg/m² to 100 mg/m² body surface area of the patient, on days 1 and 15 of a 28 day treatment cycle, over an infusion time of 60 minutes.

In embodiments of the present invention, there is provided a kit comprising tinostamustine or a pharmaceutically acceptable salt thereof or medicament comprising the same together with instructions.

The instructions may advise administering tinostamustine or a pharmaceutically acceptable salt thereof according to variables such as the state of the solid tumours being treated; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts.

In a further embodiment of the present invention, the patient in need of said treatment is given radiotherapy with (including prior to, during or after) treatment of the solid tumour(s) with tinostamustine or a pharmaceutically acceptable salt thereof. In embodiments of the present invention, the patient is treated with tinostamustine or a pharmaceutically acceptable salt thereof and radiotherapy. Preferably, the patient is given radiotherapy treatment prior to the treatment with tinostamustine or a pharmaceutically acceptable salt thereof. The radiotherapy may be given at a dose of 1 to 5 Gy over 5-10 consecutive days and preferably 2 Gy over 5-10 consecutive days.

In a further embodiment of the present invention, the patient in need of said treatment is given radiotherapy prior to or after treatment of the solid tumours with tinostamustine or a pharmaceutically acceptable salt thereof. Preferably, the patient is given radiotherapy treatment prior to the treatment with tinostamustine or a pharmaceutically acceptable salt thereof. The radiotherapy may be given at a dose of 1 to 5 Gy over 5-10 consecutive days and preferably 2 Gy over 5-10 consecutive days.

When intended for oral administration, tinostamustine or a pharmaceutically acceptable salt thereof or medicament comprising the same may be in solid or liquid form, where semi-solid, semi-liquid, suspension and gel forms are included within the forms considered herein as either solid or liquid.

Tinostamustine or a pharmaceutically acceptable salt thereof or medicament comprising the same can be prepared for administration using methodology well known in the pharmaceutical art. Examples of suitable pharmaceutical formulations and carriers are described in “Remington's Pharmaceutical Sciences” by E. W. Martin.

As a solid composition for oral administration, tinostamustine or a pharmaceutically acceptable salt thereof can be formulated into a powder, granule, compressed tablet, pill, capsule, chewing gum, wafer or the like form. Such a solid composition typically contains one or more inert diluents or carriers. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When tinostamustine or a pharmaceutically acceptable salt thereof compositions is in the form of a capsule (e.g. a gelatin capsule), it can contain, in addition to materials of the above type, a liquid carrier such as polyethylene glycol, cyclodextrin or a fatty oil.

Tinostamustine or a pharmaceutically acceptable salt thereof compositions can be in the form of a liquid, e.g. an elixir, syrup, solution, emulsion or suspension. The liquid can be useful for oral administration or for delivery by injection. When intended for oral administration, tinostamustine or a pharmaceutically acceptable salt thereof compositions can comprise one or more of a sweetening agent, preservatives, dye/colorant and flavor enhancer. In tinostamustine or a pharmaceutically acceptable salt thereof compositions for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, tinostamustine or a pharmaceutically acceptable salt thereof or medicament comprising the same is administered intravenously.

Liquid forms of tinostamustine or a pharmaceutically acceptable salt thereof or medicament comprising the same, may be solutions, suspensions or other like form, and can also include one or more of the following: sterile diluents such as water for injection, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is a preferred adjuvant.

Tinostamustine or a pharmaceutically acceptable salt thereof or medicament comprising the same can be administered by any convenient route, for example by infusion or bolus injection, by absorption through epithelial or mucocutaneous linings, and preferably by bolus.

Examples of compositions comprising tinostamustine or a pharmaceutically acceptable salt thereof are disclosed in WO2013/040286.

The present invention encompasses combinations of tinostamustine or a pharmaceutically acceptable salt and a DNA repair inhibitor.

The present invention encompasses combinations of tinostamustine or a pharmaceutically acceptable salt and a DNA damage response (DDR) inhibitor.

The present invention encompasses combinations of tinostamustine or a pharmaceutically acceptable salt and a PARP inhibitor.

The PARP inhibitor may be olaparib.

The present invention encompasses combinations of tinostamustine or a pharmaceutically acceptable salt and carboplatin. The carboplatin may be administered further in conjunction with a DNA repair inhibitor, a DDR inhibitor, a PARP inhibitor or olaparib.

In one embodiment of the combination of the present invention, tinostamustine or a pharmaceutically acceptable salt thereof and a further agent(s) are adapted for administration concurrently, sequentially or separately. Preferably, tinostamustine or a pharmaceutically acceptable salt thereof and the further agent(s) are adapted for administration concurrently.

The molar ratio of further agent to tinostamustine or a pharmaceutically acceptable salt thereof in the combination of the present invention is typically from 1:2000 to 2000:1. Preferably, the molar ratio of carboplatin to tinostamustine or a pharmaceutically acceptable salt thereof in said combination is from 1:2000 to 1:100, more preferably the molar ratio of carboplatin to tinostamustine ora pharmaceutically acceptable salt thereof in said combination is from 1:1000 to 1:500, and most preferably it is from 1:900 to 1:500, e.g. 1:900, 1:800, 1:700, 1:600 or 1:500.

The further agent may be a DNA repair inhibitor, a DDR inhibitor, a PARP inhibitor, olaparib or carboplatin.

It has been surprisingly found that combinations comprising tinostamustine or a pharmaceutically acceptable salt thereof and a further agent are synergistic combinations. In other words, the potency of the combinations has been measured with the Calcusyn software (biosoft, Ferguson, MO, USA), which is based on the Chou Talay method (Chou et al., Adv. Enzyme Regul., 22, 27-55 (1984)), that calculates a combination index (CI) with the following interpretation:
CI 1>1: antagonist effect, CI=1: additive effect and CI<1 synergistic effect.

The present invention may be further understood by consideration of the following non-limiting examples.

### EXAMPLES

In the following examples, tinostamustine is referred to as EDO-S101.

EDO-S101 may be prepared as described in Example 6 of WO-A-2010/085377.

### Example 1—Activity of EDO-S101 in a Triple Negative Breast Cancer Spheroid Model

### Materials and Methods

The activity of EDO-S101 alone and in combination was evaluated in triple negative breast cancer (TNBC) cells lines using a spheroid model.

### EDO-S101 and Control Compounds

EDO-S101 was provided by EDO MundiPharma, and synthesised as described in Example 6 of WO-A-2010/085377.
Carboplatin was purchased from Sigma.
Paclitaxel was purchased from Sigma.
Olaparib was purchased from Sigma.

### Test System

Spherotech TNBC cell line forming multicellular spheroids were prepared for the cell line shown in Table 1.

TABLE 1

| | Spheroid | Site of | His- | Molecular | TNBC | BRCA- |
|---|---|---|---|---|---|---|
| Cell line | formation | origin* | tology# | subtype | subtype | 1 status |
| HCC1143 | + | PT | IDC | Basal A | BL1 | wt |

Fresh primary triple negative breast cancer samples were obtained after informed consent of the individual patient was given. Patients were recruited following clinical diagnosis, and prior to chemotherapy commencing.

Heterotypic tumour spheroids were prepared from the individual primary breast cancer tissue. The tumour samples were mechanically and enzymatically digested using an enzyme cocktail (Roche, Penzberg Germany), cell viability determined using the trypan-blue exclusion test, and spheroids seeded at a cell density of 50,000 using a modified liquid overlay method. After 48 h in cell culture under standard conditions, successful spheroid formation was documented photographically before starting treatment.

The spheroids were treated with EDO-S101 (5 μM, 10 μM) alone and in combination with carboplatin and/or the PARP inhibitor olaparib (Table 2) for different periods of time (48, 72 h). Solvent controls and an untreated (medium) control was used under the same culture conditions. Treatment efficacy of all drugs was analysed with a standard ATP assay.

TABLE 2

| | | | Solvent | |
|---|---|---|---|---|
| Single agent | Concentration | | control | Combination therapy |
| EDO-S101 | 5 | μM | DMSO 0.25% | EDO 5 μM + Carboplatin |
| EDO-S101 | 10 | μM | DMSO 0.5% | EDO10 μM + Carboplatin |
| Carboplatin (C) | 40.84 | μg/ml | H2O 0.41% | EDO 5 μM + Olaparib |
| Paclitaxel (P) | 1.53 | μg/ml | NaCl 0.03% | EDO10 μM + Olaparib |
| Olaparib (Ola) | 6.6 | μg/ml | DMSO 0.07% | Carboplatin + Olaparib |

| | | | |
|---|---|---|---|
| Medium control | — | — | Epi + Cyclo + Paclitax |
| (untreated) | | | |

The results of the experiments are shown in Tables 3a and 3b below. The results are the mean of six repeats per treatment based on the results of the ATP assay.

TABLE 3a

| | HCC1143, p13 | |
|---|---|---|
| | mean cell | |
| | viability | |
| Therapy T48 h | * | SD # |
| EDO S101 5 μM | 85.75 | 13.19 |
| EDO S101 10 μM | 57.43 | 2.28 |
| EDO S101 5 μM + Carboplatin | 93.19 | 19.74 |
| EDO S101 10 μM + Carboplatin | 109.25 | 30.02 |
| EDO S101 5 μM + Olaparib | 53.01 | 4.74 |
| EDO S101 10 μM + Olaparib | 68.52 | 6.38 |
| EDO S101 5 μM + Carbopl. + Ola | 82.18 | 13.93 |
| EDO S101 10 μM + Carbo. + Ola | 64.32 | 3.00 |
| Carboplatin | 118.28 | 16.51 |
| Paclitaxel | 92.95 | 8.18 |
| Olaparib | 87.97 | 9.68 |
| Carboplatin + Olaparib | 92.40 | 8.27 |
| Paclitaxel + Olaparib | 85.29 | 2.44 |
| untreated | 107.92 | 2.82 |

TABLE 3b

| | HCC1143 | |
|---|---|---|
| | mean cell | |
| | viability | |
| Therapy T72 h | * | SD # |
| EDO S101 5 μM | 79.99 | 12.71 |
| EDO S101 10 μM | 77.09 | 15.36 |
| EDO S101 5 μM + Carboplatin | 93.22 | 12.43 |
| EDO S101 10 μM + Carboplatin | 82.25 | 17.83 |
| EDO S101 5 μM + Olaparib | 47.51 | 2.29 |
| EDO S101 10 μM + Olaparib | 45.22 | 8.11 |
| EDO S101 5 μM + Carbopl. + Ola | 58.15 | 4.14 |
| EDO S101 10 μM + Carbo. + Ola | 52.03 | 3.22 |
| Carboplatin | 105.88 | 8.38 |
| Paclitaxel | 85.59 | 12.72 |
| Olaparib | 61.88 | 11.65 |
| Carboplatin + Olaparib | 105.45 | 5.34 |
| Paclitaxel + Olaparib | 93.17 | 11.77 |
| untreated | 88.55 | 11.14 |

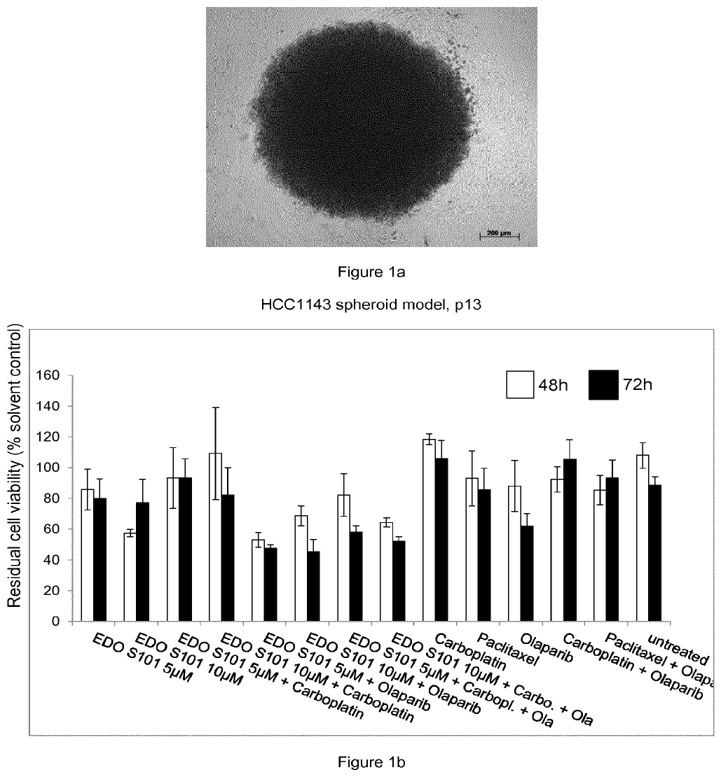

FIG. 1a shows a HCC1143 p13 spheroid 48 h after seeding, and before starting treatment. FIG. 1b shows the residual cell viability (% solvent control) of the HCC1143 p13 spheroid model following treatment with EDO-S101 alone and in combination with carboplatin and/or olaparib after 48 h and 72 h. HCC1143 cells are from the BL1 triple negative breast cancer subtype.

After 48 h, treatment with EDO-S101 (5 μm and 10 μm) alone demonstrated the most enhanced reduction in cell viability (86% and 57% residual cell viability respectively) compared to any of the control agents (carboplatin, paclitaxel, olaparib, or combinations thereof) which range from 85-118%. Treatment with 10 μm EDO-S101 revealed pronounced reduction in cell viability activity against HCC1143 cells. The most pronounced reduction in cell viability (53% residual cell viability) was observed in spheroids treated with EDO-S101 (5 μm)+Ola.

After 72 h treatment with EDO-S101 (5 μM and 10 μM) as a monotherapy, enhanced activity against spheroid cell viability (80% and 77% residual cell viability respectively) compared to the negative control was observed. In addition EDO-S101 monotherapy was more effective than treatment with paclitaxel, carboplatin, C+Ola or P+Ola, but was less effective than Ola alone (62% residual cell viability). However, EDO-S101 (5 μM and 10 μM)+Ola (45% and 48% residual cell viability respectively) demonstrated improved efficacy over Ola as a monotherapy, as did EDO-S101 (5 μM and 10 μM)+Ola+C (58% and 52% residual cell viability respectively). EDO-S101 therefore demonstrated enhanced efficacy against HCC1143 cell viability as compared against Paclitaxel, Carboplatin, Olaparib or any combinations thereof.

FIGS. 1a and 1b demonstrate that EDO-S101 alone has activity against TNBC. The figures also demonstrate that EDO-S101 in combination other cancer drugs also demonstrate activity against TNBC. This is shown in the examples in relation to:
EDO-S101 plus the platinum agent cisplatin;
EDO-S101 plus the PARP inhibitor olaparib;
EDO-S101 plus cisplatin and olaparib.

EDO-S101 is shown to have activity in the BL1 subtype.

EDO-S101 is further shown to have activity in BRCA-1 wild type TNBC, as shown from cell line HCC1143.

### Example 3

A Phase 1/2 Study to Investigate the Safety, Pharmacokinetics and Efficacy of EDO-S101, a First-in-Class Alkylating Histone Deacetylase Inhibition (HDACi) Fusion Molecule, in Patients with Advanced Solid Tumors

### Purpose

Phase 1: To determine the safety, tolerability, maximum tolerated dose (MTD), and recommended phase 2 dose (RP2D) of EDO-S101 as a single agent in patients with solid tumors who have progressed after at least one (1) line of standard therapy.
Phase 2: To evaluate the efficacy of EDO-S101 in selected tumor types.

| Condition | Intervention | Phase |
|---|---|---|
| Phase 1: | Drug: EDO-S101 | Phase 1/2 |
| Advanced or metastatic Solid Tumors | | |
| Phase 2: | | |
| small cell lung cancer (SCLC) | | |
| soft tissue sarcoma or non-Kit GIST | | |
| triple negative breast cancer | | |
| ovarian cancer | | |

Study Type: Interventional

Study Design: Intervention Model: Single Group Assignment

Masking: Open Label

Primary Purpose: Treatment

### Primary Outcome Measures:

Phase 1: Dose Escalation until maximum administered dose (MAD):
Determine Maximum Tolerated Dose at optimal infusion schedule

### Phase 2: Evaluation of Toxicity and Response Rate in Selected Solid Tumor Cohorts:

Confirm recommended Phase 2 dose and optimal infusion schedule in select solid tumors
Determine objective response rate and clinical benefit rate in selected solid tumors

### Secondary Outcome Measures:

Phase 1: Dose Escalation until MAD:
Determine maximum plasma concentration (Cmax) of EDO-S101

### Phase 2: Evaluation of Toxicity and Response Rate in Selected Solid Tumor Cohorts:

To evaluate safety and tolerability of the RP2D of the selected schedule of EDOS101.
To determine the progression free survival time for patients who received the RP2D at the optimal infusion schedule
To determine overall survival for patients who received the RP2D at the selected study drug administration schedule.
To establish the trough PK profiles of EDO-S101.

Estimated Enrollment: 158

### Phase 1:

Schedule A: EDO-S101, IV, 60 mg/m² up to 150 mg/m² Day 1 and 15 of each 28 day cycle

Schedule B: EDO-S101, IV, 60 mg/m² up to 150 mg/m² Day 1, 8 and 15 of each 28 day cycle

### Phase 2:

The RP2D and selected schedule will be further investigated in patients with specific types of solid tumors: relapsed/refractory SCLC, soft tissue sarcoma, non-Kit GIST, triple negative breast, and ovarian cancers.

### DETAILED DESCRIPTION

EDO-S101 I entity, a first-in-class fusion molecule of an alkylator, bendamustine and a histone-deacetylase inhibitor (HDACi), vorinostat. This phase 1/2 study will enroll patients with solid tumours. This phase 1/2 study will enroll patients with various advanced solid tumours.

The study consists of 2 phases:
Phase 1: Dose Escalation until MAD
Phase 2: Evaluation of Toxicity and Response Rate in Selected Solid Tumor Cohorts

The study is designed as an open label, Phase 1/2 trial of single agent EDOS101. The phase 1 portion of the study is designed to define the MTD for two (2) administration schedules by evaluating toxicities during dose escalation until MAD. The phase 2 portion of the study is designed to evaluate ORR and CBR at four (4) or six (6) months depending on the type of solid tumor.

### Eligibility

Ages Eligible for Study: 18 Years and older (Adult, Senior)

Sexes Eligible for Study: All

Accepts Healthy Volunteers: No

### Criteria

Inclusion Criteria for Phase 1 and phase 2 portions of study:

1. Signed informed consent.

2. patients≥18 years at signing the informed consent.

3. Diagnosis of advanced or metastatic solid tumors, disease should have progressed following at least one line of standard therapy.

4. Patients with secondary metastasis to the CNS are eligible if they have met certain criteria.

5. Evaluable disease; either measurable on imaging or with informative tumor marker.

6. Discontinuation of previous cancer therapies at least three (3) weeks or 5 half-lives, whichever is shorter.

7. Eastern Cooperative Oncology Group (ECOG) performance status≤2

8. Neutrophils≥1,500 μL.

9. Platelets≥100,000 μL.

10. Aspartate aminotransferase/alanine aminotransferase (AST/ALT)≤3 upper limit of normal (ULN). In cases with liver involvement ALT/AST≤5×ULN.

11. Total bilirubin≤1.5 mg/dL unless elevated due to known Gilbert's syndrome.

12. Creatinine≤1.5 ULN.

13. Serum potassium within normal range.

14. If female of child-bearing potential (i.e. not postmenopausal or surgically sterile), must be willing to abstain from sexual intercourse or employ an effective barrier or medical method of contraception during the study drug administration and follow-up periods. If male, must be sterile or willing to abstain from sexual intercourse or employ a barrier method of contraception during the study treatment and at least 6 months following last treatment.

### Exclusion Criteria for Phase 1 and Phase 2 Portions of Study:

1. Patients with primary central nervous system (CNS) cancer.

2. Patients with QTc interval>450 msec for male and >470 msec for female.

3. Patients who are on treatment with drugs known to prolong the QT/QTc interval.

4. Patients who are on treatment with Valproic Acid in any of its indication (epilepsy, mood disorder) must be excluded or must stop using the medication.

5. Any serious medical condition that interferes with adherence to study procedures.

6. Prior history of solid tumor malignancy diagnosed within the last three (3) years of study enrollment excluding adequately treated basal cell carcinoma of the skin, squamous cell carcinoma of the skin, or in situ cervical cancer, in situ breast cancer, in situ prostate cancer (patients must have shown no evidence of active disease for 2 years prior to enrollment)

7. Pregnant or breast feeding females.

8. New York Heart Association (NYHA) stage III/IV congestive heart failure, arrhythmias not adequately controlled, or other significant co-morbidities [e.g. active infection requiring systemic therapy, history of human immunodeficiency virus (HIV) infection, or active Hepatitis B or Hepatitis C].

9. Use of other investigational agents within 30 days or 5 half-lives prior to the first dose of study drug. As long as patient has recovered from any related toxicities≥Grade 1.

10. Steroid treatment within seven (7) days prior to study treatment. Patients that require intermittent use of bronchodilators, topical steroids or local steroid injections will not be excluded from the study. Patients who have been stabilized to 10 mg orally prednisolone PO QD (or equivalent), daily or less seven (7) days prior to study drug administration are allowed.

### Phase 2 Tumor-Specific Eligibility Criteria

Phase 2 patients must meet the cohort-specific inclusion/exclusion criteria in addition to the general inclusion/exclusion criteria for Phase 1 and Phase 2 study listed above.

### Cohort 1 Patient Population: Relapsed/Refractory SCLC

1. Histologically or cytological confirmed limited or extensive disease stage of SCLC. The disease should be progressing during or relapsing after the previous treatment.

2. At least one line of prior combination chemotherapy including adequate doses of platinum compound and having progressed during therapy or after the previous treatment.

3. At least 3 weeks or 5 half-lives, whichever is shorter, should have elapsed since prior treatment as long as the patient recovered from any related toxicities to ≤Grade 1.

4. Prior radiotherapy is acceptable provided the patient has recovered from any radiotherapy related acute toxicities.

### Cohort 2 Patient Population: Relapsed/Refractory Soft Tissue Sarcoma or Non-Kit GIST

1. Histologically confirmed diagnosis of advanced, unresectable, or metastatic soft tissue sarcoma not amenable to curative treatment with surgery or radiotherapy excluding: chondrosarcoma, neuroblastoma, osteosarcoma, embryonal rhabdomyosarcoma, or Kaposi sarcoma.

2. Must have received at least one prior first line combination chemotherapy regimen or at least two first line single-agent regimens. Adjuvant chemotherapy not considered first line, unless disease progression within 6 months of treatment.

3. The disease should be progressing/relapsed during or after the previous treatment. At least 3 weeks should have elapsed since prior chemotherapy or 5 half-lives, whichever is shorter, as long as the patient recovered from any related toxicities to ≤Grade 1.

4. Presence of measurable disease as defined by the Response Evaluation Criteria in Solid Tumors (RECIST 1.1, Eisenhauer et al. 2009).

### Cohort 3 Patient Population: Relapsed/Refractory Triple Negative Breast Cancer

1. Histologically or cytologically confirmed locally advanced or metastatic Triple Negative Metastatic Breast Cancer.

2. Must have received at least one line of chemotherapy, at least 3 weeks should have relapsed since prior chemotherapy or 5 half-lives, whichever is shorter, as long as the patient recovered from acute toxicity of previous therapies to ≤grade 1.

3. Prior radiotherapy is acceptable provided it was applied within 4 four weeks before starting of this trial and the patient recovered from any radiotherapy related acute toxicities.

4. The disease should be progressing/relapsed during or after the previous treatment.

5. Presence of measurable disease as defined by the Response Evaluation Criteria.

### Cohort 4 Patient Population: Relapsed/Refractory Ovarian Cancer

1. Histologically or cytologically confirmed advanced ovarian cancer: epithelial ovarian cancer, primary peritoneal cancer or fallopian tube cancer (excluding borderline ovarian cancer) that is resistant or refractory to platinum therapy.
a. Platinum-resistant ovarian cancer is defined as disease that responded to primary platinum therapy and then progressed within 6 months or disease that progressed during or within six months of completing a subsequent platinum therapy.
b. Primary platinum refractory disease is defined as disease that has not responded to a platinum-based regimen or experienced disease recurrence within 3 months of completing a first-line platinum-based regimen.

2. The disease should be progressing/relapsed during or after the previous treatment. At least 3 weeks should have elapsed since prior chemotherapy or 5 half-lives, whichever is shorter, as long as the patient recovered from acute toxicity of previous therapies to grade 1.

3. Presence of measurable disease as defined by the Response Evaluation Criteria in Solid tumors (RECIST 1.1, Eisenhauer et al. 2009).

1-22. (canceled)

23. A method of treating advanced or metastatic triple negative breast cancer (TNBC) in a patient in need thereof, comprising administering to said patient tinostamustine or a pharmaceutically acceptable salt thereof, at a dosage range from 60 mg/m² to 100 mg/m² body surface area of said patient through infusion, in combination with a radiotherapy, wherein the TNBC is basal-like 1 (BL1) subtype, BRCA-1 wildtype, or a combination thereof.

24. The method of claim 23, wherein said tinostamustine or pharmaceutically acceptable salt thereof is administered over an infusion time of about 60 minutes.

25. The method of claim 23, wherein said tinostamustine or pharmaceutically acceptable salt thereof is administered over an infusion time of about 45 minutes.

26. The method of claim 23, wherein the radiotherapy is administered concurrently.

27. The method of claim 23, wherein the radiotherapy is given to the patient prior to or after administration of said tinostamustine or pharmaceutically acceptable salt thereof.

28. The method of claim 23, wherein said tinostamustine or pharmaceutically acceptable salt thereof is administered intravenously to the patient in need thereof on days 1 , 8 and 15 of a 28 day treatment cycle, or on days 1 and 15 of a 28 day treatment cycle.

29. The method of claim 23, wherein said radiotherapy treatment is given to the patient in need thereof at a dose of 1 to 5 Gy over 5 to 10 consecutive days.

30. The method of claim 23, wherein said radiotherapy treatment is given to the patient in need thereof at a dose of 2 Gy over 5 to 10 consecutive days.

31. A method of treating advanced or metastatic triple negative breast cancer (TNBC) in a patient in need thereof, comprising administering to said patient tinostamustine or a pharmaceutically acceptable salt thereof at a dosage range from 60 mg/m² to 100 mg/m² body surface area of said patient through infusion, and one or more further agents comprising a PARP inhibitor, wherein the TNBC is basal-like 1 (BL1) subtype, BRCA-1 wildtype, or a combination thereof.

32. The method of claim 31, wherein said tinostamustine or pharmaceutically acceptable salt thereof is administered over an infusion time of about 60 minutes.

33. The method of claim 31, wherein said tinostamustine or pharmaceutically acceptable salt thereof is administered over an infusion time of about 45 minutes.

34. The method of claim 31, wherein the PARP inhibitor is olaparib.

35. The method of claim 31, wherein said tinostamustine or pharmaceutically acceptable salt thereof is administered intravenously to the patient in need thereof on days 1 , 8 and 15 of a 28 day treatment cycle, or on days 1 and 15 of a 28 day treatment cycle.

36. The method of claim 35, further comprising treating the patient with a radiotherapy treatment.

37. The method of claim 36, wherein said radiotherapy treatment is given to the patient in need thereof at a dose of 1 to 5 Gy over 5 to 10 consecutive days.

38. The method of claim 36, wherein said radiotherapy treatment is given to the patient in need thereof at a dose of 2 Gy over 5 to 10 consecutive days.
